SEQUENCE LISTING (3 sequences)

SEQ ID NO: 1 - DNA, 20 bp, Artificial
  Synthetic antisense (AS) oligodeoxynucleotides (ODN) against DDR1
cactcccaagccatccacct

SEQ ID NO: 2 - DNA, 20 bp, Artificial
  Synthetic antisense (AS) oligodeoxynucleotide (ODN) against DDR1
ctattgctccctctgttccc

SEQ ID NO: 3 - DNA, 20 bp, Artificial
  Synthetic antisense (AS) oligodeoxynucleotide (ODN) against DDR1
gtccttccagtccatccagc

(19) United States
(12) Patent Application Publication
(10) Pub. No.: US 2016/0304881 A1
(43) Pub. Date: Oct. 20, 2016

(54) DDR1 ANTAGONIST OR AN INHIBITOR OF DDR1 GENE EXPRESSION FOR USE IN THE PREVENTION OR TREATMENT OF CRESCENTIC GLOMERULONEPHRITIS

(71) Applicant: INSERM (INSTITUT NATIONAL DE LA SANTE ET DE LA RECHERCHE MEDICALE), Paris (FR)

(72) Inventors: Christos CHATZIANTONIOU, Paris (FR); Jean-Claude DUSSAULE, Paris (FR)

(21) Appl. No.: 15/200,014

(22) Filed: Jul. 1, 2016

Related U.S. Application Data
(62) Division of application No. 14/356,635, filed on May 7, 2014.
(60) Provisional application No. 61/556,361, filed on Nov. 7, 2011.

(51) Int. Cl. C12N 15/113; C07K 16/28

(52) U.S. Cl. CPC C12N 15/1138; C07K 16/28; C12N 2310/11; C07K 2317/76

(57) ABSTRACT

The present invention relates to uses, methods and compositions for treating crescentic glomerulonephritis. More specifically, the present invention relates to a DDR1 antagonist or an inhibitor of DDR1 gene expression for the prevention or the treatment of said disease.

### FIELD OF THE INVENTION

The present invention relates to uses, methods and compositions for treating crescentic glomerulonephritis. More specifically, the present invention relates to a DDR1 antagonist or an inhibitor of DDR1 gene expression for the prevention or the treatment of said disease.

### BACKGROUND OF THE INVENTION

Glomerulonephritis (GN) refers to a heterogeneous group of diseases characterized by inflammatory changes in glomerular capillaries and accompanying signs and symptoms of an acute nephritic syndrome. Among diseases of this group, Rapidly Progressive GlomeruloNephritis (RPGN), also called crescentic glomerulonephritis or extracapillary glomerulonephritis, consists of the most severe class of glomerulopathies in humans. This disease is a clinical syndrome and a morphological expression of severe glomerular injury. Glomerular injury manifests as a proliferative histological pattern, accumulation of T cells and macrophages, proliferation of intrinsic glomerular cells, accumulation of cells in Bowman's space (“crescents”), and rapid deterioration of renal function.

Infiltration of inflammatory cells and injury of resident glomerular cells lead to the dysfunction of the capillary circulation and to the formation of glomerular crescents. Extension of the disease to the tubulo-interstitial compartment induces tubular damage and progression of renal fibrosis. The functional consequences of the structural lesions of the kidneys are proteinuria, retention of sodium and rapidly progressive loss of the renal function. The pathogenesis of the disease partly remains unclear and its treatments are insufficiently effective, justifying new experimental studies to better understand the mechanisms of renal injury. Therefore, there is currently no efficient treatment to stop or reverse the course of glomerulonephritis. Thus, new methods for the treatment of such a disease that are effective and convenient are really needed. An understanding of the mechanisms of glomerulonephritis would therefore help in the development of therapeutic strategies for these diseases.

The experimental alloimmune anti-glomerular basement membrane (anti-GBM) nephritis is a model commonly used to study mechanisms of crescentic glomerulonephritis. Injection of sheep scrum rich in immunoglobulins against glomerular antigens induces an immediate inflammatory response characterized by the renal infiltration of cells of the immune system and followed by glomerular injury.

Discoidin Domain Receptor 1 (DDR1) is a tyrosine kinase transmembrane receptor of collagens, expressed in several cell types and organs, including gastro-intestinal tract, brain, lung, mammary gland and kidney (Vogel et al., 2006). Upon activation by binding to fibrillar or soluble collagens, DDR1 regulates cell differentiation, proliferation and migration. Its role during the skin wound repair or the development of inner ear and of mammary gland has been previously reported. A number of studies have shown that overexpression of this receptor was implicated in cell migration in tumors, inflammation, atherosclerosis. The implication of DDR1 in renal injury has been studied in mice by deletion of its gene. In mice, constitutive renal expression of DDR1 predominates in vascular smooth muscle cells, and to a lesser extent in glomerular cells (Flumant et al., 2006). Consistent with the important pathogen role of this receptor in renal diseases, DDR1-deficient (DDR−/−) mice are protected against renal lesions induced by a chronic infusion of angiotensin II, a model in which haemodynamic alterations and vascular remodeling play a major role (Flamant et al., 2006). Gross et al have demonstrated deleterious implication of DDR1 in a model of Alport's disease (Gross et al., 2010). More recently, we observed that renal inflammation was reduced in the tubulo-interstitial model of unilateral ureteral obstruction (UUO) (Guerrot et al., 2011).

However, until now no study provides the evidence that DDR1 interfered with the progression of crescentic glomerulonephritis.

### SUMMARY OF THE INVENTION

Now, the invention provides a new method for the treatment of crescentic glomerulonephritis.

The inventors have indeed found that in mice and humans, crescentic glomerulonephritis is associated with increased DDR1 expression in glomeruli. Inhibition of the activity of this receptor by deletion of the gene or injection of antisense (AS) oligodeoxynucleotides (ODN) considerably protects the mice against loss of renal function and death. Indeed, DDR1 deficient mice did not exhibit crescentic glomeruli despite injection of sheep nephrotoxic serum (NTS). Finally, the inventors have showed that administration of an inhibitor of DDR1 gene inhibitor (AS ODN) in wild type mice receiving NTS suppressed albuminuria and glomerular injury and prevented renal failure and death.

These data unravel a prominent pathophysiological role for the DDR1 in acute crescentic glomerulonephritis and suggest that inhibitors of the DDR1 cascade may be needed for preventing severe renal damage and renal failure.

Therefore, a first aspect or the present invention relates to a DDR1 antagonist for use in the prevention or the treatment of crescentic glomerulonephritis.

A second aspect of the present invention relates to an inhibitor of DDR1 gene expression for use in the prevention or the treatment of crescentic glomerulonephritis.

### DETAILED DESCRIPTION OF THE INVENTION

### Definitions

Throughout the specification, several terms are employed and are defined in the following paragraphs.

A “coding sequence” or a sequence “encoding” an expression product, such as an RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, references to specific proteins (e.g., DDR1) can include a polypeptide having a native amino acid sequence, as well as variants and modified forms regardless of their origin or mode of preparation. A protein that has a native amino acid sequence is a protein having the same amino acid sequence as obtained from nature (e.g., DDR1). Such native sequence proteins can be isolated from nature or can be prepared using standard recombinant and/or synthetic methods. Native sequence proteins specifically encompass naturally occurring truncated or soluble forms, naturally occurring variant forms (e.g., alternatively spliced forms), naturally occurring allelic variants and forms including postranslational modifications. A native sequence protein includes proteins following post-translational modifications such as glycosylation, or phosphorylation, or other modifications of some amino acid residues.

Variants refer to proteins that are functional equivalents to a native sequence protein that have similar amino acid sequences and retain, to some extent, one or more activities of the native protein. Variants also include fragments that retain activity. Variants also include proteins that are substantially identical (e.g., that have 80, 85, 90, 95, 97, 98, 99%, sequence identity) to a native sequence. Such variants include proteins having amino acid alterations such as deletions, insertions and/or substitutions. A “deletion” refers to the absence of one or more amino acid residues in the related protein. The term “insertion” refers to the addition of one or more amino acids in the related protein. A “substitution” refers to the replacement of one or more amino acid residues by another amino acid residue in the polypeptide. Typically, such alterations are conservative in nature such that the activity of the variant protein is substantially similar to a native sequence protein (see, e.g., Creighton (1984) Proteins, W.H. Freeman and Company). In the case of substitutions, the amino acid replacing another amino acid usually has similar structural and/or chemical properties. Insertions and deletions are typically in the range of 1 to 5 amino acids, although depending upon the location of the insertion, more amino acids can be inserted or removed. The variations can be made using methods known in the art such as site-directed mutagenesis (Carter, et al. (1986) Nucl. Acids Res. 13:4331; Zoller et al. (1987) Nucl. Acids Res. 10:6487), cassette mutagenesis (Wells et al. (1985) Gene 34:315), restriction selection mutagenesis (Wells, et al. (1986) Philos. Trans. R. Soc. London SerA 317:415), and PCR mutagenesis (Sambrook et al., Molecular Cloning: A Laboratory Manual, 3rd edition, Cold Spring Harbor Press, N.Y., (2001)).

Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical). Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs which are modified, by processes such as capping, polyadenylation, methylation, and editing, and proteins (e.g., DDR1) modified by, for example, methylation, acetylation, phosphorylation, ubiquitination, SUMOylation, ADP-ribosylation, myristilation, and glycosylation.

An “inhibitor of gene expression” refers to a natural or synthetic compound that has a biological effect to inhibit the expression of a gene.

A “receptor” or “receptor molecule” is a soluble or membrane bound/associated protein or glycoprotein comprising one or more domains to which a ligand binds to form a receptor-ligand complex. By binding the ligand, which may be an agonist or an antagonist the receptor is activated or inactivated and may initiate or block pathway signaling.

The term “DDR1” or “Discoidin domain receptor family, member 1”, also known as CD167a (cluster of differentiation 167a) refers a receptor protein tyrosine kinase (RTK) which belongs to a subfamily of RTK which possess an extracellular domain related to the lectin discoidin, found in the slime mold Dietyostelium discoideum, and that are activated by various types of collagen. All members of the subfamily share the approximately 160-amino acid-long amino terminal discoidin homology domain followed by a single transmembrane region, and extended juxtamembrane region, and a catalytic tyrosine kinase domain.

DDR1 appears in five isoforms, a (Accession No, NM_013993), b (Accession No. NM_001954), c (Accession No. NM_013994), d (Accession No. AF353182), and c (Accession No. AF353183), which are generated by alternative splicing (all GenBank entries are incorporated by reference).

By “ligand” or “receptor ligand” is meant a natural or synthetic compound which binds a receptor molecule to form a receptor-ligand complex. The term ligand includes agonists, antagonists, and compounds with partial agonist/antagonist action.

An “agonist” or “receptor agonist” is a natural or synthetic compound which binds the receptor to form a receptor-agonist complex by activating said receptor and receptor-agonist complex, respectively, initiating a pathway signaling and further biological processes.

By “antagonist” or “receptor antagonist” is meant a natural or synthetic compound that has a biological effect opposite to that of an agonist. An antagonist binds the receptor and blocks the action of a receptor agonist by competing with the agonist for receptor. An antagonist is defined by its ability to block the actions of an agonist.

The term “DDR1 antagonist” refers to any DDR1 antagonist that is currently known in the art or that will be identified in the future, and includes any chemical entity that, upon administration to a patient, results in inhibition of a biological activity associated with activation of the DDR1 in the patient, including any of the downstream biological effects otherwise resulting from the binding to DDR1 of its natural ligand. Such DDR1 antagonist includes any agent that can block DDR1 activation or any of the downstream biological effects of DDR1 activation. Such an antagonist can act by binding directly to the intracellular domain of the receptor and inhibiting its kinase activity. Alternatively, such an antagonist can act by occupying the ligand binding site or a portion thereof of the DDR1 receptor, thereby making the receptor inaccessible to its natural ligand so that its normal biological activity is prevented or reduced. Thus, a DDR1 antagonist may for instance block or inhibit DDR1 activation or phosphorylation (e.g., blocking or inhibiting collagen-induced tyrosine phosphorylation of DDR1).

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

By “purified” and “isolated” it is meant, when referring to a polypeptide (i.e. DDR1) or a nucleotide sequence, that the indicated molecule is present in the substantial absence of other biological macromolecules of the same type. The term “purified” as used herein preferably means at least 75% by weight, more preferably at least 85% by weight, still preferably at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

### Therapeutic Methods and Uses

The present invention provides for methods and compositions (such as pharmaceutical compositions) for treating crescentic glomerulonephritis.

Thus, a first aspect of the present invention relates to a DDR1 antagonist for use in the prevention or the treatment of crescentic glomerulonephritis.

In one embodiment, the DDR1 antagonist may be a low molecular weight antagonist.

Low molecular weight DDR1 antagonists are well known in the art. For example, low molecular weight DDR1 antagonists that may be used by the invention include, for example pyrimidylaminobenzamide DDR1 antagonists and thienopyridine DDR1 antagonists as well as all pharmaceutically acceptable salts and solvates of said DDR1 antagonists, such as those described in the following patent publications: International Patent Publication Nos. WO 2011/062927, WO 2011/050120 and WO 2010/062038.

Additional non-limiting examples of low molecular weight DDR1 antagonists include any of the Bcr-Abl tyrosine kinase inhibitors (such as imatinib, dasatinib, and nilotinib) since these three inhibitors have also been described in Day et al. (2008) as inhibitors of collagen-induced DDR1 activation.

Therefore, a specific example of low molecular weight DDR1 antagonist that can be used according to the present invention may be the (4-(4-methylpiperazin-1-yhnethyl)-N-[4-methyl-3-(4-pyridin-3-yl)pyrimidin-2-ylamino)phenyl-benzamide) (also known as ST1571, imatinib or GLIVEC®; Novartis) (International Patent Publication No. WO 95/09852) Another specific example of a low molecular weight DDR1 antagonist that can be used according to the present invention may be the 4-methyl-N-[3-(4-methyl-1H-imidazol-1-yl)-5-(trifluoromethyl)phenyl]-3-[(4-pyridin-3-ylpyrimidin-2-yl) amino]benzamide (also known as AMN107, nilotinib or TASIGNA®; Novartis) (International Patent Publication No. WO 2004/005281).

Another specific example of a low molecular weight DDR1 antagonist that can be used according to the present invention may be the N-(2-chloro-6-methylphenyl)-2-[[6-[4-(2-hydroxyethyl)-1-piperazinyl]-2-methyl-4-pyrimidinyl]amino]-5-thiazole carboxamide (also known as BMS-354825, dasatinib or SPRYCEL®; Bristol-Myers Squibb) (International Patent Publication No. WO 2004/085388)

In a particular embodiment, said low molecular weight DDR1 antagonist is selective. Low molecular weight DDR1 antagonists are small organic molecules, said antagonists are preferably selective for the DDR1 receptor as compared with the other tyrosine kinase receptors, such as EGF receptor. By “selective” it is meant that the affinity of the antagonist for the DDR1 is at least 10-fold, preferably 25-fold and more preferably 100-fold higher than the affinity for the other tyrosine kinase receptors (such as EGF receptor).

In another embodiment, the DDR1 antagonist may consist in an antibody or antibody fragment that can partially or completely block or inhibit DDR1 activation or phosphorylation (e.g., blocking or inhibiting collagen-induced tyrosine phosphorylation of DDR1).

Non-limiting examples of antibody-based DDR1 antagonists include those described in International Patent Publication No. WO 2010/019702. Thus, the DDR1 antagonist can be the monoclonal antibody Mab 20M 102 (ATCC Accession No. PTA-10051) or an antibody or antibody fragment having the binding specificity thereof (that specifically binds to a particular extracellular domain of human DDR1 described in said International Patent Publication.

Additional antibody antagonists can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against DDR1 can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al, 1983); and the EBV-hybridoma technique (Cole et al, 1985, Monoclonal Antibodies and Cancer Therapy, Alan R. Liss, Inc., pp. 77-96). Alternatively, techniques described for the production of single chain antibodies (see, e.g., U.S. Pat. No. 4,946,778) can be adapted to produce anti-DDR1 single chain antibodies. DDR1 antagonists useful in practicing the present invention also include anti-DDR1 antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed to allow rapid identification of fragments having the desired specificity to DDR1.

Humanized anti-DDR1 antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fe), typically that of a human immunoglobulin. Methods for making humanized antibodies are described, for example, by Winter (U.S. Pat. No. 5,225,539) and Boss (Celltech, U.S. Pat. No. 4,816,397).

Another aspect of the invention relates to an inhibitor of DDR1 gene expression for use in the prevention or the treatment of crescentic glomerulonephritis.

Inhibitors of DDR1 gene expression for use in the present invention may be based on antisense oligonucleotide constructs. Anti-sense oligonucleotides, including anti-sense RNA molecules and anti-sense DNA molecules, would act to directly block the translation of DDR1 mRNA by binding thereto and thus preventing protein translation or increasing mRNA degradation, thus decreasing the level of DDR1 proteins, and thus activity, in a cell. For example, antisense oligonucleotides of at least about 15 bases and complementary to unique regions of the mRNA transcript sequence encoding DDR1 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In one embodiment, the sequence of the anti-sense oligonucleotide targeting DDR1 is represented by SEQ ID NO: 1.

In one embodiment, the sequence of the anti-sense oligonucleotide targeting DDR1 is represented by SEQ ID NO: 2.

In one embodiment, the sequence of the anti-sense oligonucleotide targeting DDR1 is represented by SEQ ID NO: 3.

Small inhibitory RNAs (siRNAs) can also function as inhibitors of DDR1 gene expression for use in the present invention. DDR1 gene expression can be reduced by contacting the tumor, subject or cell with a small double stranded RNA (dsRNA), or a vector or construct causing the production of a small double stranded RNA, such that DDR1 gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Specific examples of siRNAs targeting DDR1 that can be used according to the present invention include those described in the US Patent Publication No. US 2007/255048

Ribozymes can also function as inhibitors of DDR1 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucicolytic cleavage. Engineered hairpin or hammerhead motif ribozyme molecules that specifically and efficiently catalyze endonucicolytic cleavage of DDR1 mRNA sequences are thereby useful within the scope of the present invention. Specific ribozyme cleavage sites within any potential RNA target are initially identified by scanning the target molecule for ribozyme cleavage sites, which typically include the following sequences, GUA, GuU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides, siRNAs and ribozymes useful as inhibitors of DDR1 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SPG polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating, the transfer of the antisense oligonucleotide, siRNA or ribozyme nucleic acid to the cells and preferably cells expressing DDR1. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses For certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. Sec e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Another aspect of the invention relates to a method for treating crescentic glomerulonephritis comprising administering a patient in need thereof with a therapeutically effective amount of an antagonist or inhibitor of gene expression as above described.

In the context of the invention, the term “treating” or “treatment”, as used herein, means reversing, alleviating, inhibiting the progress of, or preventing the disorder or condition to which such term applies, or one or more symptoms of such disorder or condition.

According to the invention, the term “patient” or “patient in need thereof” is intended for a human or non-human mammal affected or likely to be affected with crescentic glomerulonephritis.

By a “therapeutically effective amount” of the antagonist or inhibitor of gene expression as above described is meant a sufficient amount of the antagonist or inhibitor of gene expression to treat crescentic glomerulonephritis at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Screening Methods

Antagonists of the invention can be further identified by the screening methods described in the state of the art. The screening methods of the invention can be carried out according to known methods.

The screening method may measure the binding of a candidate compound to the receptor, or to cells or membranes bearing the receptor, or a fusion protein thereof by means of a label directly or indirectly associated with the candidate compound. Alternatively, a screening method may involve measuring or, qualitatively or quantitatively, detecting the competition of binding of a candidate compound to the receptor with a labelled competitor (e.g., antagonist or agonist). Further, screening methods may test whether the candidate compound results in a signal generated by an antagonist of the receptor, using detection systems appropriate to cells bearing the receptor. Antagonists can be assayed in the presence of a known agonist (e.g., collagen) and an effect on activation by the agonist by the presence of the candidate compound is observed. Further, screening methods may comprise the steps of mixing a candidate compound with a solution comprising DDR1, to form a mixture, and measuring the activity in the mixture, and comparing to a control mixture which contains no candidate compound. Competitive binding using known agonist such collagen is also suitable.

### Pharmaceutical Compositions

The antagonists or inhibitors of gene expression of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transderm al, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The antagonist or inhibitor of expression of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylaminc, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The antagonist or inhibitor of expression of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### Example

### Material & Methods

Animals: Female transgenic mice and their wild type (wt) littermates aged 3 to 6 months and weighing 18 to 25 g were used in these experiments. Wt and DDR1−/− mice were bred in our own facilities onto a Sv129 background (Flamant et al., 2006). These mice have been backcrossed 8 times to 129/Sv. For the experiments with AS administration, Sv129 mice (3 to 6 months old) were purchased from Janvier (Le Genest-St-Isle, France). Decomplemented sheep nephrotoxic serum (NTS) was prepared as described previously (Mesnard et al., 2009). Crescentic glomerulonephritis was induced in 18 wt and 18 DDR1−/− mice by intravenous administration of a total 23 μl NTS/g body weight, administered over three consecutive days (days 0, 1 and 2). Concentrations of sheep IgG in mouse serum at d8 were 1.50±0.13 (n=5) and 1.62±0.25 ng/ml (n=5; NS), respectively in wt and DDR1−/− mice (Sheep IgG, Sigma Diagnostics). Animals were sacrificed 4, 8 or 17 days following serum administration. Sham kidneys were used as controls (n=6). In a separate series of experiments examining mortality rates (n−10 wt and n=10 DDR1−/−), experiments were terminated at the 42^{nd} day. In AS experiments, animals (n=10 mice with glomerulonephritis, n=13 mice with glomerulonephritis receiving scrambled ODN, n=17 mice with glomerulonephritis receiving specific AS, n=8 control mice receiving scrambled ODN or AS) were sacrificed 2 weeks after NTS administration. Overall, 102 mice were used for the present study (68 wt and 34 DDR1−/−). All mice were kept in well-controlled animal housing facilities and had free access to tap water and pellet food. All animal procedures were in accordance with the European Guidelines for the Care and use of Laboratory Animals.

Proteinuria and BUN: All mice were acclimated in metabolic cages with free access to food and water for 24-hour urine collection. Proteinuria was assessed using the Pyrogallol Red method, utilizing a KONELAB automate (Thermo Scientific, Waltham, Mass.), and expressed as g protein/mmol creatininuria. Urea concentration (BUN) was assessed in blood plasma obtained on the day of sacrifice, using an enzymatic-spectrophotometric method and was expressed in mmol/L.

Blood Pressure: Systolic blood pressure was measured with the CODA mouse/rat tail cuff system (Kent Scientific Corporation). Animals were accustomed for several days before measurements were made. To avoid variations in blood pressure due to day cycle, all measurements were carried between 14.00 and 16.00 h. Only animals that did not display signals of stress and that showed stable and reproducible values of blood pressure for at least three consecutive days were considered for blood pressure measurements. Ten measurements from each mouse were taken at two minutes intervals then a mean value was determined.

Assessment of anti-sheep IgG titers in mouse serum: Anti-sheep IgG titers were measured in serum of mice by ELISA assay (Alpha Diagnostic International). Plates were coated with 20 μg/ml sheep IgG (Alpha Diagnostic) overnight at 4° C., and then blocked using a 5% albumin solution. Serum to be tested was added to the wells at various dilutions according to the manufacturer's instructions. Each sample was assayed in duplicate.

Masson's trichrome staining: Kidneys were fixed in alcohol-formalin-acetic acid, embedded in paraffin, cut into 3-μm sections, and stained with Masson's trichromic solution. Crescent formation was defined as glomeruli exhibiting two or more layers of cells in Bowman's space, with or without podocyte injury, as indicated by ballooning, necrosis, or cyst formation (Mesnard et al., 2009). The proportion of glomeruli affected was determined by examining a minimum of 50 glomeruli per mouse. Tubular dilations and cell infiltration were scored on a scale of 0 to 4. Scoring was performed in a blinded manner on coded slides.

Sirius red morphometric analysis: Interstitial fibrosis was assessed on 8 μm-thick Sirius red-stained paraffin sections at 40× magnification, under polarized light. Interstitial fibrosis was quantified using computer-based morphometric analysis software (Axionplan, Axiophot2, Zeiss, Germany). Twelve cortical fields excluding interlobular arteries were selected randomly from each kidney. Data were expressed as the mean value of the percentage of positive area examined.

Martins Scarlet Blue staining: Kidneys were fixed in alcohol-formalin-acetic acid, embedded in paraffin, cut into 3-μm sections, and stained. Fibrin deposits appear in red color. The percentage of glomeruli presenting fibrin deposits was determined by examining at least 50 glomeruli per mouse.

Immunohistochemistry and immunofluorescence in mice: Four-micrometers-thick cryostat sections of renal cortex were fixed with acetone for 7 min, After blockade of endogenous peroxidase, they were stained with anti-CDR (Santa Cruz Biotechnology, Santa Cruz, Calif.) or anti-F4/80 (AbCys, Paris, France) and the Envision kit (DakoFrance, Trappes, France) was applied for 30 min at room temperature. Staining was revealed by applying DAB kit (Dako), hematoxylin QS (Vector, Burlingame, Calif.) and Permanent Mounting Media Aqueous based (Innovex, Richmond, Va.). For semi-quantitative analysis of CD3- and F4/80-positive cells, slides were independently examined on a blinded basis, using a 0- to 4-point relative intensity scale. Indexes from individual sections were averaged to calculate a global index for each kidney. Immunofluorescent experiments were performed using frozen sections fixed in acetone and then washed with PBS and incubated with anti-DDR1 (C-20, Santa Cruz Biotechnology,), anti-nephrin (H300; Santa Cruz Biotechnology), anti-rabbit FITC and anti-rabbit TRITC (Jackson Immunoresearch, West Grove, Pa.). Immunofluorescence micrographs were obtained using an Olympus BX 51 camera DP70 (Olympus, Rungis, France).

Immunohistochemistry for DDR1 in humans: Renal biopsies from patients were retrospectively analyzed. Informed consent was given by the patients for use of part of the biopsy for scientific purpose. All procedures and use of tissue were performed according to the national ethical guidelines and were in accordance with the declaration of Helsinki. Cellular crescents contained three or more layers of cells without interposition of extracellular matrix. Five biopsies from patients with rapidly progressive glomerulonephritis were examined, two lupus nephritis cases and three Goodpasture's syndrome cases. Controls consisted of normal portions of kidney removed during surgery for renal carcinoma (two biopsies) and patients with minimal change disease (three cases). Immunochemistry for DDR1 was performed in parafine sections as described in the previous paragraph.

qRT-PCR on podocytes in culture and renal cortex: RNA was extracted from podocytes using EZ Spin columns (Fermentas, Saint Leon-Rot, Germany) and from renal cortex using TRI REAGENT (Euromedex, Mundolsheim, France). After digestion with DNase 1, RNA was reverse transcribed with Maxima RT Kit (Fermentas). The cDNA obtained was then amplified by PCR in a LightCycler 480 (Roche Diagnostics, Meylan, France) with SYBR Green (Fast Start DNA Master SYBR® Green I, Roche Diagnostics) and specific primers for target mRNAs designed using the Universal Probe Library Roche website under the following conditions: 95° C. for 5 min, 45 cycles at 95° C. for 15 s and 60° C. for 15 s, and 72° C. for 15 s. PCR was also carried out for two housekeeping genes: β1-actin and β-Glucuronidase B (GUS B). Results are expressed as 2^{deltaCp}, where Cp is the cycle threshold number normalized to the mean 2^{deltaCp} for each corresponding control group. Dissociation curves were analyzed after each run for each amplicon in order to determine the specificity of quantification when using SYBR Green.

Administration of antisense (AS) against DDR1: To block DDR1 expression, we used a cocktail of 3 specific AS oligodeoxynucleotides (ODN) designed on IDT DNA (Integrated DNA Technologies) modified with phosphorothioate to prevent their in vivo hydrolysis by nucleases (Sigma Aldrich, St Quentin Fallavier, France). The absence of cross reactivity with related sequences in GenBank was checked. The AS or scrambled control ODNs were diluted in 0.9% sodium chloride solution and administrated by intraperitoneal injections every 48 hours (100 μmol/ODN/injection) with a pre-injection 48 hours before the first injection of the nephrotoxic scrum (NTS). In addition, two groups of control mice (without NTS) received the AS or scrambled ODNs.

Isolation of glomeruli: Kidneys from Sv129 mice were obtained eight days after the first injection of NTS scrum with AS or scrambled administration. Glomeruli were extracted using the following sieving procedure: kidneys were dissected then digested in a solution of collagenase (Type 1, Gibco BRL Invitrogen, Cergy-Pontoise, France, at 1 mg/ml in RPMI) at 37° C. for 3 minutes. After addition of RPM′ 10% Fetal calf scrum (Biowest, Abcys, Paris, France), the solution was passed through a 100 μm cell strainer (BD Biosciences, Le pont de Claix, France) and glomeruli were separated and washed with PBS buffer containing 0.5% of BSA to avoid aggregation (Bovine Serum Albumin, Fraction V, Euromedex) on a 40 μm cell strainer. Contamination with tubular fragments was less than 10% as assessed by phase contrast microscopy. Glomeruli were then collected by centrifugation at 1500 rpm for 3 min.

Western blot analysis: Proteins were extracted from renal cortex or isolated glomeruli using RIPA lysis buffer supplemented with sodium orthovanadate, PMSF, a protease inhibitor cocktail (Tebu bio, Le Perray en Yvelines, France) and sodium fluorure 10 mM. After centrifugation at 10 000 rpm for ten minutes at 4° C., protein concentrations were determined from the supernatant using the Bradford assay. Aliquots of 20 μg of protein were run on NuPAGE 4/12% electrophoresis gels (Invitrogen) then transferred on a PVDF membrane (Immobilon-p, Millipore, St Quentin en Yvelines, France). Immunoblotting was performed using rabbit specific primary antibodies anti-nephrin H300 (Santa Cruz) and rabbit anti-beta actin (Imgenex, San Diego, Calif., USA) for loading control. Then, the membrane was incubated with horseradish peroxidase-linked donkey secondary antibody (GE Healthcare Life Sciences, Saclay, France). The revelation was performed with the ECL plus kit (GE Healthcare). Densitometric analysis on Image J was then performed for quantification.

Podocyte culture: A previously described conditionally immortalized mouse podocyte cell line (Mundel et al., 1997) was maintained in RPMI 1640 (GIBCO) supplemented with 10% fetal bovine scrum, 100 U/ml penicillin/streptomycin (GIBCO BRL) and 10 U/mL recombinant mouse γ-interferon (Peprotech) to induce synthesis of the immortalizing T antigen in humidified incubators with air-5% CO. Subcultivation was done with trypsin at 37° C. after cells had reached confluence. To initiate differentiation, cells were thermoshifted to 37° C. and maintained in medium without γ interferon for one week. After 8 h of incubation with Heparin-binding EGF-like growth factor (HB-EGF; 50 ng/ml), transforming growth factor beta-1 (TGFβ1; 2 ng/ml), IL-1 beta (10 ng/ml) and soluble collagen type I (Coll; 100 μg/ml), the cells were harvested and total RNA was extracted.

Statistical Analysis: Quantitative analyses of histology and immunostaining were carried out using blinded coded slices. Statistical analyses were performed using analysis of variance followed by Fisher's Protected Least Significance Difference test. Survival analysis was calculated using Kaplan-Meier method (Statview Software, SAS Institute). Results with P<0.05 were considered statistically significant. All values are means±SEM.

Results

Activation of the DDR1 gene during crescentic glomerulonephritis was observed in parallel to changes in expression of tuphrin in podocytes: DDR1 mRNA measured by qRT-PCR was increased in kidneys after injection of NTS in wt mice. The difference with baseline was significant 4 days after induction of the glomerulonephritis (p<0.05) and reached a 17-fold increase at day 17 (d17) (p<0.001). The predominant renal localization of DDR1 in control mice was the vascular wall as evidenced by immunochemistry. In contrast, during crescentic glomerulonephritis, this expression was mainly observed in glomeruli and more precisely in podocytes as shown by comparison with the immunolocalization of nephrin. Apart from its de novo expression, extra-glomerular staining of DDR1 was essentially vascular, as in controls.

The functional severity of crescentic glomerulonephritis was attenuated in DDR1−/− mice: Crescentic glomerulonephritis induced hypertension, body weight increase due to sodium retention with ascites, and proteinuria in wt NTS-injected mice. Systolic blood pressure significantly rose at d8 (p<0.001 vs. controls). Body weight dramatically increased ten days after induction of glomerulonephritis and was associated with ascites and elevated proteinuria. In parallel, we observed a reduced glomerular filtration rate, reflected by a progressive increase in BUN (p<0.01). Functional parameters were identical in basal conditions between wt and DDR1−/− mice but differed significantly after induction of glomerulonephritis. When DDR1 gene was deleted, systolic blood pressure did not rise at d8, and body weight and proteinuria increase were blunted compared to wt NTS-injected mice. BUN levels increased only at d17 but to a lesser extent than in wt mice (16±6 vs. 28±14 mmol/L; p<0.05). Interestingly, the difference in blood pressure persisted between both groups at d27 (123±II in DDR1−/−vs. 157±19 mmHg in wt mice; p<0.05). The deleterious role of DDR1 expression was confirmed during the chronic phase of the disease with a percentage of survival that remained unchanged in DDR1−/− from d28 to d45 (70%) while it progressively diminished down to 10% during the same period in wt (logrank p<0.05).

The structural severity of crescentic glomerulonephritis was attenuated in DDR1−/− mice: Injection of NTS induced severe histological alterations in the kidneys of wt mice. When the macroscopic aspect of kidneys was studied at d17 after NTS injection, they appeared less colored than those of control animals. Microscopic examination revealed that glomeruli with crescent formation reached 23±9% of all glomeruli at d17 and that tubular dilations increased with time. These renal damages were significantly attenuated in DDR1−/− mice (p<0.05). Macroscopic aspect of kidneys from these mice was intermediate between those of controls and wt mice injected with NTS. Crescent formation was 2-fold diminished at d4, d8 and d17 (p<0.05) and tubular dilation was reduced in renal sections, especially at d17 (p<0.01).

Fibrin deposition is one of the key components of glomerular injury in crescentic glomerulonephritis. Martius Scarlet Blue staining demonstrated less fibrin deposits in glomeruli of DDR1−/− NTS-injected mice than in wt at the three periods of histological examinations (p<0.05). These results indicate that DDR1−/− mice were partially protected against glomerular thrombi. Because de novo synthesis of plasminogen activator inhibitor-1 (PAI-1) is implied in thrombotic process, we measured its renal mRNA expression in control conditions and after induction of the disease. As expected, PAI-1 mRNA increased several fold during the disease. In contrast, PAI-1 was barely increased in DDR1−/− mice with a highly significant difference of stimulation between both groups (p<0.01).

Role of DDR1 in the inuntoto-inflanunatory response associated with crescentic glomerulonephritis: Because antibody deposition may participate in the development of the disease, we assessed the humoral response of DDR1−/− and wt mice to sheep IgG. Similar titers of mouse anti-sheep antibodies were observed in both groups. Thus, there was no evidence that DDR1 altered the humoral immune response in this model. In addition, wt and DDR1−/− mice displayed similar CD3-positive T cells infiltrates around the glomeruli and the vessels 17 days after serum injection although there was a trend towards a difference between both groups earlier in the progression of the disease. Fewer F4/80-positive macrophages were observed in the kidney cortex of DDR1−/− than of wt mice and this difference reached a statistical difference on d17 after NTS (p<0.05). These results explain the decreased index of cell infiltration, studied on Trichrome-stained renal sections, in DDR1−/− mice compared to wt at d17 (0.87±0.17 vs. 2.21±0.88, respectively; p<0.01).

Inflammatory mediators differed between both NTS groups. IL-1 beta, a major pro-inflammatory cytokine in this model, was induced by NTS and was significantly blunted in DDR1−/− mice compared to wt (p<0.01). Similarly, expressions of three mediators involved in the recruitment of inflammatory cells, monocyte chemotactic protein-1 (MCP-1) and inter-cellular and vascular cell adhesion molecules (ICAM-1 and VCAM-1) were blunted in DDR1−/−.

Role of DDR1 in the fibrotic response associated with crescentic glonzeridonephritis: We next assessed the effect of DDR1 deletion on the development of renal fibrosis. Seventeen days after the induction of glomerulonephritis, Sirius red score showed a 5-fold increase in the renal cortex of wt mice injected with NTS, compared to control kidneys. DDR1−/− mice presented a 33% reduction in the accumulation of fibrillar collagen compared to wt mice (p<0.01). This histological result was confirmed by qRT-PCR evaluation of col Iα2 and col IIIα1 mRNA in these groups of mice. mRNA expressions of col 1Vα3 and TGF beta1, a key pro-fibrotic agent, were also significantly lower in DDR1−/−, consistent with reduced fibrogenesis.

Consequences ref DDR1 blockade by specific AS ODN: To overcome the renal and potentially vascular consequences of DDR1 gene deletion during the development of mice, we performed additional studies in wt mice treated by specific AS ODN directed against DDR1 mRNA. Doses and sequences were validated in preliminary experiments. This group of mice was compared, after 2 weeks, to controls and to two supplementary groups of NTS-injected mice, receiving or not scrambled ODN. Scrambled ODN administration did not modify the course of the renal disease in NTS mice. AS treatment blunted the increase of DDR1 expression. Although DDR1 mRNA inhibition was partial (−56%), the beneficial effects of the specific AS-treatment were similar to those observed in DDR1−/− mice. The localization of DDR1 did not differ, in renal cortex between AS and scrambled ODN mice. In control mice receiving AS, expression of DDR1 was observed in vascular cells where as in NTS mice, its expression was predominant in glomerular cells. As in previous experiments in DDR1−/− mice, we observed a functional and a structural protection against glomerulonephritis in AS-treated mice. Proteinuria, body weight increase and BUN levels were intermediate in this group compared to those of control mice and of mice receiving scrambled ODN. Macroscopic aspect of kidneys differed between both groups and glomeruli and tubular injuries were attenuated in AS-treated mice with less crescents and tubular dilation, respectively. Fibrin deposits in glomeruli and renal expression of PAI-1 mRNA were very low in AS-treated mice compared to mice receiving scrambled ODN. Interestingly, we confirmed the protection against alteration of podocyte phenotype when DDR1 synthesis was inhibited. Nephrin expression, studied by qRT-PCR from renal cortex and by Western Blot from isolated glomeruli at d15 was improved in AS-treated compared to mice receiving scrambled ODN (densitometry ratio of nephrinlbeta actin in controls: 1.8±0.4 (n=8); in NTS±scrambled ODN: 0.45±0.09 (n=6; p<0.01 vs. controls); in NTS+AS ODN: 1.08±0.39 (n=5; not significant vs. controls). In addition, podocin expression remained unchanged in AS-treated animals, whereas it was deeply decreased (p<0.01) in NTS-injected mice without AS. Because HB-EGF production by podocytes seems to play a major role in the migration of these cells when they participate to the formation of crescents, we tested the effect of DDR1 inhibition on this growth factor mRNA. In the absence of AS treatment, HB-EGF mRNA was highly stimulated 2 weeks after NTS injection compared to basal values (p<0.01) while in AS-treated mice, this stimulation was less marked (p<0.05) although still significant compared to controls (p<0.05).

The role of DDR1 in the immuno-inflammatory and fibrotic responses in crescentic glomerulonephritis was confirmed in these experiments. Results obtained at d15 in AS-treated mice were similar to those previously described in DDR1−/− mice. Titers of mouse anti-sheep antibodies and evaluation of CD3-positive cells were identical with or without AS, whereas F4/80-positive cells markedly differed between both groups as well as evaluation of fibrillar collagen deposit by Sirius red staining (p<0.01). mRNA expressions of IL-1 beta, MCP-1, ICAM-1, VCAM-1 (not shown), TGF beta1, col lag and col Mal were blunted in mice treated by AS.

In vitro experiments in cultured podocytes: To confirm the interaction between DDR1 expressed in podocytes and the immuno-inflammatory process during crescentic glomerulonephritis, we performed in vitro experiments in highly differentiated cultured podocytes. DDR1 mRNA, evaluated by qRT-PCR increased in presence of IL-1 beta and collagen I (p<0.05), whereas it did not differ from basal values in presence of FIB-EGF or TGF beta I.

DDR1 is expressed in human crescentic glonterulonephritis: To test whether DDR1 expression was associated with glomerular discuses in humans, we examined biopsies of rapidly progressive glomerulonephritis from 3 patients with Goodpasture's syndrome and 2 patients with lupus nephritis. In both cases, DDR1 was expressed in glomeruli, especially in crescents when they were visible, while in control biopsies, the staining was on vessels and not in glomeruli.

### REFERENCES

Throughout this application, various references describe the state of the art to which this invention pertains. The disclosures of these references are hereby incorporated by reference into the present disclosure.
Vogel W F, Abdulhussein R, Ford C E: Sensing extracellular matrix: an update on discoidin domain receptor function. Cell Signal 2006, 18:1108-1116
Guerrot D, Kerroch M, Placier S, Vandermeersch S, Trivin C, Macl-Ainin M, Chatziantoniou C, Dussaule J C: Discoidin Domain Receptor 1 is a Major Mediator of Inflammation and Fibrosis in Obstructive Nephropathy. Am J Pathol 2011 May 13
Flamant M, Placier S, Rodenas A, Curat C A, Vogel W F, Chatziantoniou C, Dussaule J C: Discoidin domain receptor 1 null mice are protected against hypertension-induced renal disease, J Am Soc Nephrol 2006, 17: 3374-3381
Gross O, Girgert R, Beirowki B, Kretzler M, Kang H G, Kruegel J, Miosge N, Busse A C, Seuerer S, Vogel W F, Muller G A, Weber M: Loss of collagen-receptor DDR1 delays renal fibrosis in hereditary type IV collagen disease, Matrix Biol 2010, 29:346-356
Mesnard L, Keller A C, Michel M L, Vandermeersch S, Rafat C, Letavemier E, Tillet Y, Rondeau E, Leite-de-Moracs M C: Invariant natural killer T cells and TGF-beta attenuate anti-GBM glomerulonephritis: J Am Soc Nephrol 2009, 20:1282-1292
Mundel P, Reiser J, Zuniga Mejia Borja A, Pavenstadt H, Davidson G R, Kriz W, Zeller R: Rearrangements of the cytoskeleton and cell contacts induce process formation during differentiation of conditionally immortalized mouse podocyte cell lines. Exp Cell Res 1997, 236: 248-258.
Day E, Waters B, Spiegel K, Alnadaf T, Manley P W, Buchdunger E, Walker C, Jarai G; Inhibition of collagen-induced discoidin domain receptor 1 and 2 activation by imatinib, nilotinib and dasatinib; Eur J Pharmacol. 2008 Dec. 3; 599 (1-3):44-53.

1. A method of preventing or treating crescentic glomerulonephritis in a patient in need thereof, comprising
administering to the patient a therapeutically effective amount of a a Discoidin Domain Receptor 1 (DDR1) antagonist.

2. The method according to claim 1, wherein said DDR1 antagonist is an anti-DDR1 antibody.

3. A method of preventing or treating crescentic glomerulonephritis in a patient in need thereof, comprising
administering to the patient a therapeutically effective amount of an inhibitor of DDR1 gene expression.

4. The method according to claim 3, wherein said inhibitor of DDR1 gene expression is a siRNA, a ribozyme, or an antisense oligonucleotide.
